(19) United States
(12) Patent Application Publication
(10) Pub. No.: US 2026/0096958 A1
(43) Pub. Date: Apr. 9, 2026

(54) LIPOSOME COMPOSITION COMPRISING CULTURE EXTRACT OF SPHINGOMONAS OLEI

(71) Applicant: HUGEL INC, Chuncheon-si (KR)

(72) Inventors: Won Woo CHOI, Seoul (KR); Min Ji PARK, Seoul (KR); Byong Jun CHOI, Seoul (KR); Chi Hyun PARK, Ansan-si (KR); Jang Ho PARK, Seoul (KR); Yung Jae KIM, Seoul (KR)

(21) Appl. No.: 19/405,846

(22) Filed: Dec. 2, 2025

Related U.S. Application Data
(63) Continuation-in-part of application No. PCT/KR2023/022018, filed on Dec. 29, 2023.

(30) Foreign Application Priority Data
Jun. 3, 2023 (KR) 10-2023-0071923

(51) Int. Cl. A61K 8/14; A61K 8/02; A61K 8/34; A61K 8/37; A61K 8/49; A61K 8/55; A61K 8/99; A61Q 19/00

(52) U.S. Cl. CPC A61K 8/14; A61K 8/0245; A61K 8/345; A61K 8/37; A61K 8/4973; A61K 8/553; A61K 8/99; A61Q 19/00; A61K 2800/413

(57) ABSTRACT

The present disclosure relates to a liposome composition including a culture extract of Sphingomonas olei and a cosmetic composition including the same. The liposomal composition according to the present disclosure encapsulates an extract of Sphingomonas olei culture broth in liposome, thereby efficiently delivering the culture extract of Sphingomonas olei into the skin and thus exhibiting enhanced skin regeneration efficacy and improved transdermal absorption.

### PRIORITY CLAIM

This application is a bypass continuation-in-part application of PCT Application No. PCT/KR2323/022018, filed Dec. 29, 2023, which claims priority to Korean Patent Application No. 10-2023-0071923, filed Jun. 2, 2023, both of which are herein incorporated by reference in their entirety.

### TECHNICAL FIELD

The present disclosure relates to a liposome composition including a culture extract of Sphingomonas olei and a cosmetic composition including the same. The liposomal composition according to the present disclosure encapsulates an extract of Sphingomonas olei culture broth in liposome, thereby efficiently delivering the culture extract of Sphingomonas olei into the skin and thus exhibiting enhanced skin regeneration efficacy and improved transdermal absorption.

### BACKGROUND ART

The surface of human skin contains many waste products such as keratin, sweat, and sebum. Accordingly, various microorganisms inhabit and form the skin microbiome, which is the skin commensal flora. The skin commensal flora consists of beneficial and harmful bacteria in symbiosis, and healthy skin has a greater distribution of beneficial bacteria, while atopic or acne-prone skin has a greater distribution of harmful bacteria. Therefore, it is known that maintaining the balance of commensal bacteria existing on the skin helps maintain healthy skin condition.

Skin commensal bacteria that normally reside on the skin form a skin barrier that prevents the invasion of other more harmful microorganisms (pathogens) and assist in skin immune function. Skin beneficial bacteria do not cause disease and do not stimulate the immune system. Accordingly, if pharmaceuticals or cosmetics can be manufactured using skin beneficial bacteria, they could be used with confidence without side effects such as skin irritation.

When the skin is injured or infected, damage to skin tissue or inflammation occurs, but if the degree is not severe, the skin usually heals itself. Sphingomonas olei has been reported as one of the skin commensal bacteria that helps in the process of skin regeneration and recovery, and healing of inflammation.

Meanwhile, liposomes are spherical vesicles composed of phospholipids, and since phospholipids are the main components of biological membranes, the phospholipid membrane of liposomes also exhibits physiological functions and characteristics similar to biological membranes. Therefore, since liposomes are skin-friendly and have excellent safety, they are applied as effective drug delivery vehicles in the pharmaceutical and cosmetic industries.

Sphingomonas olei has efficacy in improving skin conditions such as skin regeneration, moisturizing, and anti-inflammation, but in the case of the culture extract of Sphingomonas olei, the application thereof is not easy due to limitations in absorption into the skin caused by the physical protective barrier of the human skin barrier when simply applied.

### DETAILED DESCRIPTION OF THE INVENTION

### Technical Problems

Accordingly, the present disclosure provides a liposome composition with enhanced transdermal absorption rate and skin regeneration efficacy by efficiently delivering the culture extract of Sphingomonas olei into the skin through encapsulation of the culture extract of Sphingomonas olei in liposomes.

In addition, the present disclosure provides a cosmetic composition including the liposome composition.

### Solution to Problem

One aspect of the present disclosure provides a liposome composition including 60 to 94 vol % of a culture extract of Sphingomonas olei, 5 to 30 vol % of glycerin, 0.05 to 10 vol % of hydrogenated lecithin, 0.005 to 5 vol % of cetearyl olivate, and 0.005 to 5 vol % of sorbitan olivate.

In the present disclosure, Sphingomonas olei may be viable bacteria (living bacteria) or dead bacteria. More specifically, the dead bacteria may be dead bacteria by heat treatment.

In an embodiment, Sphingomonas olei may be Sphingomonas olei CBN003 strain (accession No. KACC 81169BP).

In this specification, the term “culture broth” may refer to the entire medium containing the microorganism, its metabolites, and remaining nutrients obtained by culturing Sphingomonas olei for a certain period of time in a medium capable of supplying nutrients so that Sphingomonas olei can grow and survive in a culture device such as a test tube or fermenter. Additionally, the culture broth may refer to a culture broth which is obtained by removing microbial cells from the cell culture resulting from culturing microorganisms. Meanwhile, the liquid obtained by removing the cells from the culture broth is also referred to as a “supernatant,” which may be obtained by leaving the culture broth still for a certain time and taking only the upper liquid layer while excluding the portion settled at the bottom, or by removing the cells through filtration, or by centrifuging the culture broth and collecting only the upper liquid phase while removing the precipitate at the bottom. The “microbial cells” refer to the microorganisms themselves of the present disclosure, and includes microorganisms themselves isolated and selected from skin samples, etc., or microorganisms separated from culture broth after the microorganisms isolated and selected from skin samples, etc. are cultured. The microbial cells may be obtained by centrifuging the culture broth and taking the portion settled at the bottom layer, or may be obtained by leaving the culture broth still for a certain time and then removing the upper liquid since the microbial cells settle to the bottom layer of the culture broth by gravity.

According to one aspect, the culture of Sphingomonas olei of the present disclosure may use a medium easily selected by those skilled in the art from media used for microbial culture according to the purpose, and specifically, media used for Sphingomonas culture, such as reinforced clostridium medium (RCM) medium, tryptic soy broth (TSB), or brain heart infusion (BHI) medium, can be used, but is not limited thereto. According to an embodiment, the culture of Sphingomonas olei of the present disclosure may be prepared by inoculating Sphingomonas olei into the microbial culture medium and using a microbial culture method known in the art (e.g., static culture, etc.).

The culture broth may include the culture broth itself obtained by culturing a microorganism, or a concentrate or lyophilizate of the culture broth, or a culture supernatant obtained by removing a microorganism from the culture broth, or a concentrate or lyophilizate of the culture supernatant.

The culture broth may be obtained by culturing Sphingomonas olei in an appropriate medium (e.g., R2A medium or TSB medium) at a temperature of 10° to 40° C. for a certain period of time, for example, 4 to 50 hours.

The concentrate of the microorganism may be obtained from the microorganism culture broth itself, or by concentrating the supernatant obtained after centrifugation of the culture broth or filtration of the culture broth using a filter.

A culture medium and culture conditions for culturing Sphingomonas olei can be appropriately selected or modified by a person of ordinary skill in the art.

In this specification, the term “culture extract” may be used with the same meaning as “fermented extract”. The culture extract may be obtained by removing microorganisms by centrifuging or filtering the fermented culture broth. In addition, the culture extract includes those obtained by extracting from the culture broth or the concentrate thereof, and may include an extract solution, a diluted or concentrated form of the extract solution, a dried product obtained by drying the extract solution, or a crude or purified preparations thereof, and fractions obtained by fractionating the foregoing materials.

The culture extract of Sphingomonas olei may be prepared according to a conventional method in the art.

The culture extract of Sphingomonas olei may have one or more skin condition improvement effects selected from the group consisting of skin barrier improvement, skin moisturization, skin immunity improvement, skin defense enhancement, skin inflammation suppression, skin soothing, and skin regeneration (see Korean Patent Publication No. 2361015).

Culture extracts of Sphingomonas olei may promote the migration of keratinocytes on the damaged skin surface and enhance the recovery of damaged epidermis. Culture extracts of Sphingomonas olei may increase the expression of skin barrier-related factors (e.g., loricrin, a protein required for keratin formation). Additionally, culture extracts of Sphingomonas olei may reduce the expression of inflammatory factors, such as atopy and pruritus-related factors (e.g., Thymic stromal lymphopoietin (TSLP)) and proinflammatory cytokines (e.g., IL-6, IL-8 or IL-1a).

According to the present disclosure, when a culture extract of Sphingomonas olei is formulated into a liposome composition, skin regeneration may be promoted and transdermal absorption may be improved compared to a non-liposome composition.

The term “liposome” used herein refers to having the form of liposomes used in the pharmaceutical, cosmetic, and food fields to stably deliver bioactive components and maximize penetration effects.

The term “encapsulation” used herein refers to capsulation to surround the delivery substance and efficiently incorporate the same into the living body.

The liposome composition according to the present disclosure includes 60 to 94 vol % of a culture extract of Sphingomonas olei, 5 to 30 vol % of glycerin, 0.05 to 10 vol % of hydrogenated lecithin, 0.005 to 5 vol % of cetearyl olivate, and 0.005 to 5 vol % of sorbitan olivate.

In an embodiment, the liposome composition of the present disclosure may include 65 to 90 vol % of a culture extract of Sphingomonas olei, 8 to 25 vol % of glycerin, 0.1 to 7 vol % of hydrogenated lecithin, 0.05 to 4 vol % of cetearyl olivate, and 0.05 to 4 vol % of sorbitan olivate.

In an embodiment, the liposome composition of the present disclosure may include 70 to 85 vol % of a culture extract of Sphingomonas olei, 13 to 25 vol % of glycerin, 0.2 to 5 vol % of hydrogenated lecithin, 0.1 to 3 vol % of cetearyl olivate, and 0.1 to 3 vol % of sorbitan olivate.

The liposome composition of the present disclosure may include a culture extract of Sphingomonas olei in an amount of 60 to 94 vol %, 65 to 90 vol %, or 70 to 85 vol %.

The liposome composition of the present disclosure may include glycerin as a stabilizer. The liposome composition of the present disclosure may include glycerin in an amount of 5 to 30 vol %, 8 to 25 vol %, 13 to 25 vol %, or 15 to 25 vol %. In the present disclosure, when the content of glycerin exceeds 30 vol %, stickiness may occur during the manufacture of cosmetics.

The liposome composition of the present disclosure may include hydrogenated lecithin as a surfactant in an amount of 0.05 to 10 vol %, 0.1 to 7 vol %, or 0.2 to 5 vol %. Lecithin refers to a mixture of various phospholipids, and the composition of the phospholipids may vary depending on their origin. Hydrogenated lecithin may be obtained by adding hydrogen to lecithin. When hydrogenated lecithin is included in an amount less than 0.05 vol % or more than 10 vol %, liposome formation may not occur properly.

The liposome composition of the present disclosure may include cetearyl olivate and sorbitan olivate as auxiliary surfactants. Cetearyl olivate is an ester of cetearyl alcohol and the fatty acids of olive oil. Sorbitan olivate is an ester of hexitol anhydride derived from sorbitol and fatty acids of olive oil.

The liposome composition of the present disclosure may include sorbitan olivate in an amount of 0.005 to 5 vol %, 0.05 to 4 vol %, or 0.1 to 3 vol %. Additionally, the liposome composition of the present disclosure may include cetearyl olivate in an amount of 0.005 to 5 vol %, 0.05 to 4 vol %, or 0.1 to 3 vol %.

In the present disclosure, cetearyl olivate and sorbitan olivate may form a lipid bilayer of liposomes together with hydrogenated lecithin. In the present disclosure, when cetearyl olivate and sorbitan olivate are each included in an amount less than 0.005 vol %, liposome formation may not occur properly, and when included in an amount exceeding 5 vol %, the efficacy of the culture extract of Sphingomonas olei may become insufficient as the amount of components encapsulated in the liposomes decreases due to excess membrane components.

The liposome composition of the present disclosure essentially includes a culture extract of Sphingomonas olei, glycerin, hydrogenated lecithin, cetearyl olivate, and sorbitan olivate, but may additionally include components such as a liposome stabilizer, an antioxidant, etc., as needed, within a range that does not affect the essential properties of the liposome composition.

In the liposome composition of the present disclosure, the particle diameter of the liposome may be 25 to 300 nm, for example, 50 to 200 nm.

Another aspect of the present disclosure provides a cosmetic composition including the liposome composition.

The cosmetic composition may have one or more skin condition improvement effects selected from the group consisting of skin barrier improvement, skin moisturization, skin immunity improvement, skin defense enhancement, skin inflammation suppression, skin soothing, and skin regeneration (see Korean Patent Publication No. 2361015).

The cosmetic composition includes the liposome composition according to the present disclosure in an amount of 0.1 to 20 vol %, 0.1 to 15 vol %, or 0.1 to 10 vol %. In the present disclosure, when the cosmetic composition includes less than 0.1 vol % of the liposome composition, the effect of the culture extract of Sphingomonas olei may be minimal, and even when the cosmetic composition includes more than 20 vol %, the effect according to the culture extract of Sphingomonas olei can no longer be expected to increase in proportion to the amount added, making it economically undesirable.

The cosmetic composition according to the present disclosure may include components commonly used in cosmetic compositions, such as metal ion sequestrants, active ingredients (for example, sodium hyaluronate and tocopheryl acetate, etc.), preservatives, thickeners and conventional adjuvants such as fragrances, and carriers.

In addition, the cosmetic composition according to the present disclosure may be manufactured in the form of formulations common in the art, for example, emulsion formulations or solubilization formulations. Examples of emulsion formulations include nourishing lotions, creams, essences, etc., and examples of solubilization formulations include softening lotions. Additionally, the cosmetic composition of the present disclosure may be manufactured in the form of adjuvants that can be applied topically or systemically, commonly used in the dermatological field, by containing dermatologically acceptable media or bases in addition to cosmetics.

Suitable cosmetic formulations may be, for example, solutions, gels, solid or paste anhydrous products, emulsions obtained by dispersing oil in water, suspensions, microemulsions, microcapsules, microgranules or ionic (liposomes) or non-ionic vesicular dispersions, creams, skins, lotions, powders, ointments, sprays or conceal sticks. Additionally, it can be manufactured in the form of a foam or an aerosol composition containing further compressed propellant.

In addition, the cosmetic composition of the present disclosure may further include adjuvants commonly used in the cosmetic or dermatological field, such as fatty substances, organic solvents, solubilizers, thickeners and gelling agents, emollients, antioxidants, suspending agents, stabilizers, foaming agents, fragrances, surfactants, water, ionic or nonionic emulsifiers, fillers, metal ion sequestrants and chelating agents, preservatives, vitamins, blocking agents, wetting agents, essential oils, dyes, pigments, hydrophilic or lipophilic active agents, lipid vesicles, or any other components commonly used in cosmetics. These components may be introduced in amounts generally used in the dermatological field.

In the present disclosure, the cosmetic composition may be formulated as, for example, skin, lotion, body lotion, cream, essence, blemish balm (BB) cream, etc., but is not limited thereto.

Specific formulations of the cosmetic composition of the present disclosure include skin lotion, skin softener, skin toner, astringent, lotion, milk lotion, moisture lotion, nourishing lotion, massage cream, nourishing cream, moisture cream, hand cream, essence, nourishing essence, pack, soap, shampoo, cleansing foam, cleansing lotion, cleansing cream, body lotion, body cleanser, emulsion, pressed powder, loose powder, patch, spray, etc.

### Advantageous Effects of Invention

A liposome composition including a culture extract of Sphingomonas olei according to one aspect of the present disclosure exhibits enhanced skin regeneration efficacy and transdermal absorption rate by efficiently delivering the culture extract of Sphingomonas olei into the skin through encapsulation of the culture extract of Sphingomonas olei in liposomes.

### BRIEF DESCRIPTION OF THE DRAWINGS

FIG. 1 is a photograph of a liposome composition of a culture extract of Sphingomonas olei according to an embodiment of the present disclosure observed using a field emission transmission electron microscope (FE-TEM).

FIG. 2 is a graph showing the results of measuring the cell viability of human keratinocytes (HaCaT) according to the concentration % (v/v) of a liposome composition (Liposome) of a culture extract of Sphingomonas olei according to an embodiment of the present disclosure and a non-liposome composition (Non-liposome) of a culture extract of Sphingomonas olei according to an embodiment of the present disclosure, using a WST-1 assay. The untreated group was used as the control, and cell viability was expressed as a % compared to the control.

FIG. 3 is an experimental result showing the migration of human keratinocytes (HaCaT) according to the concentration % (v/v) of a liposome composition (Liposome) of a culture extract of Sphingomonas olei according to an embodiment of the present disclosure and a non-liposome composition (Non-liposome) of a culture extract of Sphingomonas olei according to an embodiment of the present disclosure. The remaining gap due to cell migration 48 hours after sample treatment is shown compared to the scratch gap of 0 hours. (N-CNT, negative control; P-CNT, positive control; #, significant compared to N-CNT value; *, significant compared to non-liposome composition sample treatment value; #p<0.005; ##, ** p<0.0005; ###, *** p<0.00005)

FIG. 4 is a photograph showing the migration of human keratinocytes according to the concentration % (v/v) of a liposome composition of a culture extract of Sphingomonas olei according to an embodiment of the present disclosure and a non-liposome composition of a culture extract of Sphingomonas olei according to an embodiment of the present disclosure. (N-CNT, negative control; P-CNT, positive control; SFE, non-liposome composition of the culture extract of Sphingomonas olei; L-SFE, liposome composition of the culture extract of Sphingomonas olei)

FIG. 5 is a result showing the depth of transdermal absorption over time for a liposome composition (Liposome) of the culture extract of Sphingomonas olei according to an embodiment of the present disclosure and a non-liposome composition (Non-liposome) of the culture extract of Sphingomonas olei according to an embodiment of the present disclosure.

### BEST MODE FOR CARRYING OUT THE INVENTION

Hereinafter, the present disclosure will be described in more detail through examples. However, these examples are intended to exemplify the present disclosure, and the scope of the present disclosure is not limited by these examples.

### Example 1. Preparation of the Culture Extract of Sphingomonas olei

Sphingomonas olei CBN003 strain (accession No. KACC 81169BP) was cultured in TSB (Tryptic soy broth) medium at 34° C. with stirring, and the strain culture broth and microbial cells were collected at the final point in the division phase (Log phase) of the growth curve. The collected strain culture broth and microbial cells were centrifuged at 6,000 rpm for 30 minutes, then sterilized by filtration through a 0.2 μm filter to prepare a culture extract.

### Example 2. Preparation of Liposome Composition of the Culture Extract of Sphingomonas olei

The culture extract of Sphingomonas olei prepared according to Example 1 and glycerin, hydrogenated lecithin, cetearyl olivate, and sorbitan olivate were mixed in the ratios shown in Table 1 and injected into a microfluidizer to prepare a liposome composition under conditions of 10 to 20° C. and 500 to 1500 bar. Evaluation and activity analysis described below were performed using the composition within one day after preparation.

TABLE 1

| Ingredients | Content (volume %) |
|---|---|
| Culture extract of Sphingomonas olei | 78.0 |
| Glycerin | 20.0 |
| Hydrogenated lecithin | 1.0 |
| Cetearyl olivate | 0.5 |
| Sorbitan olivate | 0.5 |
| Total | 100.0 |

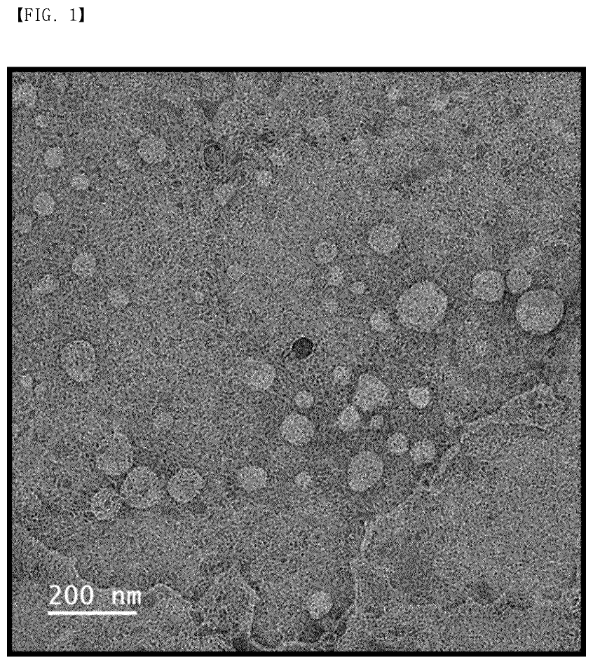

A photograph obtained by observing the prepared composition with field emission transmission electron microscopy (FE-TEM) is shown in FIG. 1, from which it can be confirmed that liposome particles were well formed.

### Comparative Example 1. Preparation of Non-Liposome Compositions of Culture Extract of Sphingomonas olei

As shown in Table 2 below, a non-liposome composition was prepared by adding purified water to the culture extract of Sphingomonas olei prepared in Example 1 without using any other components for forming liposomes. Evaluation and activity analysis described below were performed using the composition within one day after preparation.

TABLE 2

| Ingredients | Content (volume %) |
|---|---|
| Culture extract of Sphingomonas olei | 78.0 |
| Glycerin | 0 |
| Hydrogenated lecithin | 0 |
| Cetearyl olivate | 0 |
| Sorbitan Olivate | 0 |
| Distilled water | 22.0 |
| Total | 100.0 |

### Experimental Example 1. Cytotoxicity Evaluation

Whether the liposome composition of the culture extract of Sphingomonas olei prepared in Example 2 has toxicity to cells was confirmed through water soluble tetrazolium-1 (WST-1) analysis.

Human epidermal keratinocyte cell line (HaCaT) was cultured in Dulbecco's modified eagle medium (DMEM) containing 10% fetal bovine serum (FBS), 100 mg/mL streptomycin, and 100 U/mL penicillin in a 5% CO₂ incubator at 37° C. The cultured cell line was dispensed into a 24-well plate at 2×10⁵/well, cultured for 24 hours under the same conditions, and then treated with liposome compositions at concentrations of 1, 2, 5, and 10% (v/v) and non-liposome compositions at concentrations of 1, 2, 5, and 10% (v/v), and further cultured for 24 hours under the same conditions. Thereafter, WST-1 solution was added to each cell culture well and further cultured for 1 hour under the same conditions, then absorbance was measured at 450 nm.

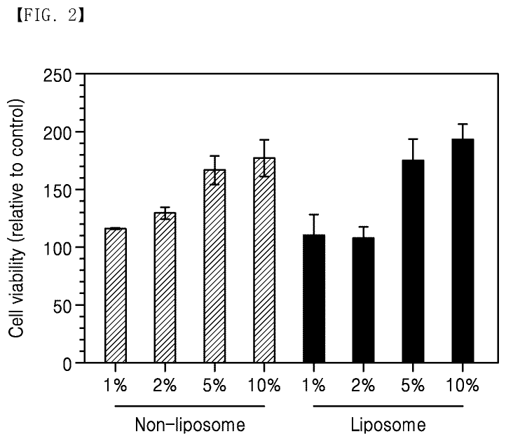

As a result, the cell viability (%) of human keratinocytes (HaCaT) compared to the control was shown in FIG. 2, using the untreated sample group as a control. As can be seen from the experimental results, when treated with the liposome composition prepared in Example 2, the cell viability of human keratinocytes (HaCaT) compared to the control was 100% or more, indicating that there was no toxicity to cells.

### Experimental Example 2. Skin Regeneration Efficacy Activity Analysis

The skin regeneration efficacy of the liposome composition of the culture extract of Sphingomonas olei prepared in Example 2 was compared with the skin regeneration efficacy of the non-liposome composition of the culture extract of Sphingomonas olei prepared in Comparative Example 1, and results thereof were analyzed.

Human keratinocyte cell line (HaCaT) was cultured in DMEM medium containing 10% FBS, 100 mg/mL streptomycin, and 100 U/mL penicillin in a 5% CO₂ incubator at 37° C. The cultured cell lines were seeded at 2.5×10⁶/well in a 6-well plate and cultured under the same conditions for 24 hours to allow cell attachment. After further culturing in serum-free DMEM medium for 24 hours, linear scratches of equal intervals were made on the cell monolayer, and the cells were cultured in serum-free DMEM medium containing 1, 2, 5, 10% (v/v) of liposome composition and 1, 2, 5, 10% (v/v) of non-liposome composition, at 37° C. in a 5% CO₂ incubator. As a positive control, cells were cultured in the same manner with 1 ng/ml of epidermal growth factor (EGF). The scratch intervals at 0 and 48 hours after sample treatment were observed under a microscope and compared and analyzed.

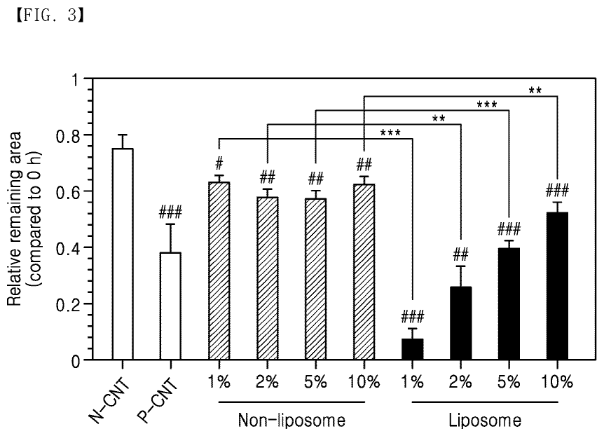

FIG. 3 shows experimental results showing the migration of human keratinocytes (HaCaT) according to the concentration % (v/v) of the liposome composition (Liposome) and non-liposome composition (non-liposome) of the culture extract of Sphingomonas olei. The remaining gap due to cell migration 48 hours after sample treatment is shown compared to the scratch gap of 0 hours. (N-CNT, negative control; P-CNT, positive control; #, significant compared to N-CNT value; *, significant compared to non-liposome composition sample treatment value; #p<0.005; ##, ** p<0.0005; ###, *** p<0.00005)

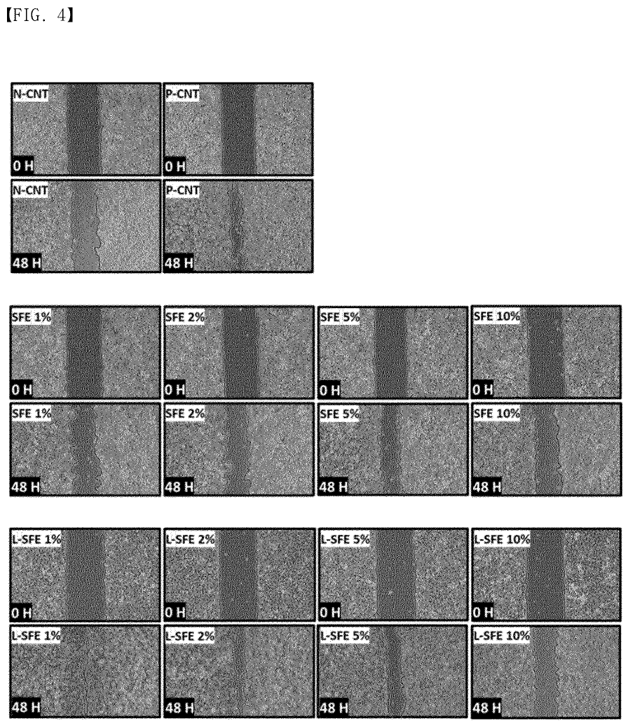

FIG. 4 is a photograph showing the migration of human keratinocytes according to the concentration % (v/v) of the liposome composition and non-liposome composition of the culture extract of Sphingomonas olei. (N-CNT, negative control; P-CNT, positive control; SFE, non-liposome composition of the culture extract of Sphingomonas olei; L-SFE, liposome composition of the culture extract of Sphingomonas olei)

As can be seen from the results, in both cases of treatment with the liposome composition and non-liposome composition of the culture extract of Sphingomonas olei, the scratch interval decreased after 48 hours of culture compared to the scratch interval at 0 hours, but it was confirmed that the scratch interval was significantly reduced in the case of treatment with the liposome composition compared to the case of treatment with the non-liposome composition sample. In particular, treatment with 1% and 2% liposome compositions was more effective in reducing scratch gaps than the positive control. These results indicate that the liposome composition of the Sphingomonas fermented extract has superior skin regeneration efficacy compared to the non-liposome composition.

### Experimental Example 3. Analysis of Transdermal Absorption Degree

The transdermal absorption degree of the liposome composition of the culture extract of Sphingomonas olei prepared in Example 2 was compared with the transdermal absorption degree of the non-liposome composition of the culture extract of Sphingomonas olei prepared in Comparative Example 1, and results thereof were analyzed.

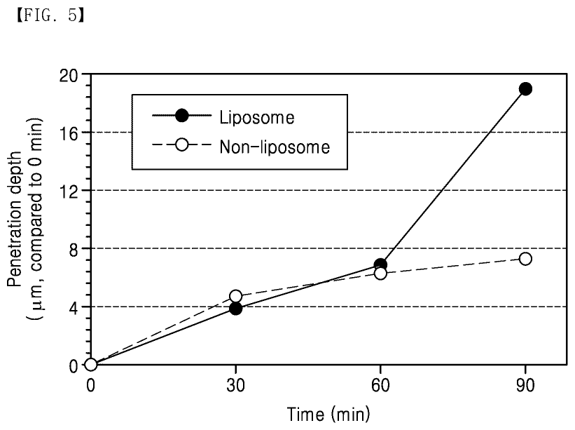

To measure the degree of transdermal absorption, a fluorescent reagent (Texas Red™, excitation wavelength of 562 nm/emission wavelength of 582-618 nm) was mixed into the strain culture broth, and samples of liposome compositions and non-liposome compositions were prepared. The samples were loaded onto Franz cell membrane (APURES), which is a test skin derived from minipigs, and the depth of absorption was measured using a fluorescence microscope after 0, 30, 60, and 90 minutes. The depth of penetration over time was compared with the depth of penetration immediately after loading (0 minutes), and the results were presented in the graph of FIG. 5.

As shown in FIG. 5, the skin penetration depth of the liposome composition of the culture extract of Sphingomonas olei measured after 30 and 60 minutes was almost the same as that of the non-liposome composition of the culture extract of Sphingomonas olei. However, after 90 minutes, the liposome composition of the culture extract of Sphingomonas olei was absorbed much more than the non-liposome composition, and the penetration depth of the liposome composition (19 μm) was about 2.6 times greater than the penetration depth of the non-liposome composition (7.17 μm). These results indicate that the liposome composition of the culture extract of Sphingomonas olei significantly improves the transdermal absorption compared to the non-liposome composition.

We claim:

1. A liposome composition, comprising
60 to 94 vol % of a culture extract of Sphingomonas olei,
5 to 30 vol % of glycerin,
0.05 to 10 vol % of hydrogenated lecithin,
0.005 to 5 vol % of cetearyl olivate, and
0.005 to 5 vol % of sorbitan olivate.

2. The liposome composition of claim 1, wherein the liposome composition comprises
65 to 90 vol % of a culture extract of Sphingomonas olei,
8 to 25 vol % of glycerin,
0.1 to 7 vol % of hydrogenated lecithin,
0.05 to 4 vol % of cetearyl olivate, and
0.05 to 4 vol % of sorbitan olivate.

3. The liposome composition of claim 1, wherein the liposome composition comprises
70 to 85 vol % of a culture extract of Sphingomonas olei,
13 to 25 vol % of glycerin,
0.2 to 5 vol % of hydrogenated lecithin,
0.1 to 3 vol % of cetearyl olivate, and
0.1 to 3 vol % of sorbitan olivate.

4. The liposome composition of claim 1, wherein the liposome composition is for promoting skin regeneration and improving transdermal absorption.

5. The liposome composition of claim 1, wherein a liposome diameter is from 25 to 300 nm.

6. A cosmetic composition comprising the liposome composition according to claim 1.

7. The cosmetic composition of claim 6, wherein the cosmetic composition comprises 0.1 to 20 vol % of the liposome composition.

8. The cosmetic composition of claim 7, wherein the cosmetic composition comprises 0.1 to 15 vol % of the liposome composition.

9. The cosmetic composition of claim 8, wherein the cosmetic composition comprises 0.1 to 10 vol % of the liposome composition.

10. The cosmetic composition of claim 6, wherein the cosmetic composition is formulated in the form of a skin, lotion, body lotion, cream, essence, or blemish balm (BB) cream.

11. The cosmetic composition of claim 6, wherein the liposome composition comprises
65 to 90 vol % of a culture extract of Sphingomonas olei,
8 to 25 vol % of glycerin,
0.1 to 7 vol % of hydrogenated lecithin,
0.05 to 4 vol % of cetearyl olivate, and
0.05 to 4 vol % of sorbitan olivate.

12. The cosmetic composition of claim 6, wherein the liposome composition comprises
70 to 85 vol % of a culture extract of Sphingomonas olei,
13 to 25 vol % of glycerin,
0.2 to 5 vol % of hydrogenated lecithin,
0.1 to 3 vol % of cetearyl olivate, and
0.1 to 3 vol % of sorbitan olivate.

13. The cosmetic composition of claim 6, wherein the liposome composition is for promoting skin regeneration and improving transdermal absorption.

14. The cosmetic composition of claim 6, wherein a liposome diameter is from 25 to 300 nm.
